(19) United States
(12) Patent Application Publication
(10) Pub. No.: US 2011/0237606 A1
(43) Pub. Date: Sep. 29, 2011

(54) 3-Deazaneplanocin Derivatives

(75) Inventors: Christina L. L. Chai, Jurong Island (SG); Eric K.W. Tam, Jurong Island (SG); Haiyan Yang, Jurong Island (SG); Qiang Yu, Genome (SG); Tuan Minh Nguyen, Jurong Island (SG)

(73) Assignee: AGENCY OF SCIENCE, TECHNOLOGY AND RESEARCH

(21) Appl. No.: 13/121,180

(22) PCT Filed: Sep. 25, 2009
(86) PCT No.: PCT/SG09/00356

§ 371 Date: Jun. 6, 2011

(30) Foreign Application Priority Data
Sep. 26, 2008 (US) 61100291

(51) Int. Cl. A61K 31/52; C07D 473/00; C07D 471/04; A61K 31/437; A61P 35/00

(52) U.S. Cl. 514/263.1; 544/264; 546/118; 514/303

(57) ABSTRACT

This invention describes the series of compounds based on the 3-deazaneplanocin A (DZNep) core structure designed to inhibit the function of Polycomb repressive complex 2 (PRC2) proteins.

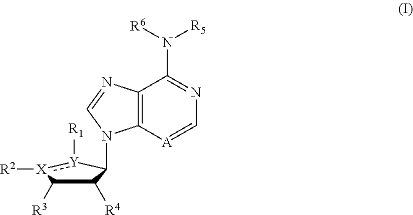

### TECHNICAL FIELD

The present invention relates to synthesis and use of 3-deazaneplanocin derivatives.

### BACKGROUND OF THE INVENTION

Cancer epigenetic regulation involves a complex biological process including DNA methylation and histone modifications, such as histone deacetylation and methylation. Small molecules targeting epigenetic process such as histone deacetylation are emerging as new classes of anti-cancer agents with promising results in clinical studies. In 2006, a histone deacetylase inhibitor (HDI), Vorinostat (also called SAHA), was approved for treatment of cutaneous T cell lymphoma (a type of skin cancer). In addition to histone deacetylation, histone methylations also play an important role in cancer epigenetics. In particular, histone methylation induced by Polycomb group (Pcg) proteins such as EZH2, which is overexpressed in multiple human cancers, is believed to be part of a mechanism causing oncogenesis and is thus an attractive target for drug development. However, no small molecules have been previously shown to inhibit this important oncogenic signalling pathway.

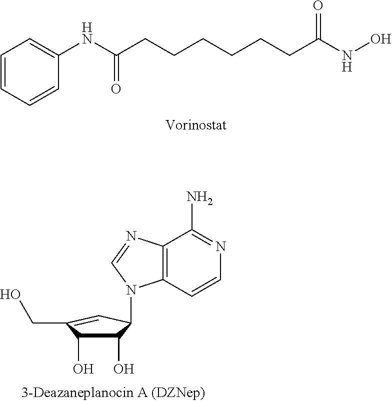

An S-adenosylhomocysteine (SAM) hydrolase inhibitor 3-Deazaneplanocin A (DZNep), which can efficiently inhibit EZH2 complex and associated H3K27 trimethylation, leading to strong apoptosis of cancer cells but not in normal cells, was recently discovered (Tan, J., Yang, X. et al. and Yu, Q. Pharmacologic disruption of Polycomb repressive complex 2-mediated gene repression selectively induces apoptosis in cancer cells. Genes & Development, 21, 1050-1063 (2007)). This discovery establishes the proof of concept that chemical inhibition of EZH2 and the associated histone methylations may represent a promising novel approach for cancer treatment. Furthermore, DZNep behaves synergistically with histone deacetylase (HDAC) inhibitors to induce apoptosis of cancer cells through effective reversal of malignant chromatin modifications. In particular, this combination treatment results in marked inhibition of Wnt/β-catenin signalling pathway in colon cancer cells, suggesting that the combination of DZNep with HDAC inhibitors may provide an effective epigenetic treatment for human cancer.

DZNep has provided promising results for both in vitro and in vivo studies. However, DZNep itself may not be an ideal drug candidate as it has a short half-life and poor bioavailability. Therefore, a new DZNep-like compound with better bioavailability is needed.

### OBJECT OF THE INVENTION

It is the object of the present invention to substantially overcome or at least ameliorate one or more of the above disadvantages. It is a further object to at least partially satisfy the above need.

### SUMMARY OF THE INVENTION

In a first aspect of the invention there is provided a compound of structure I:

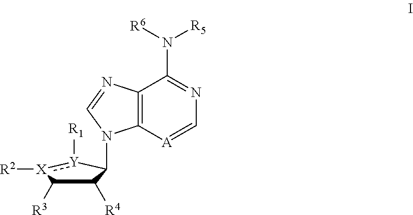

wherein:
X and Y are independently C or O;

### A is C or N;

[symbol] is a single bond or a double bond;
R¹ and R² are independently either absent or selected from the group consisting of hydrogen, halogen, optionally substituted alkyl, optionally substituted aryl, optionally substituted alkyl-Z— and optionally substituted aryl-Z—, where Z is N, O, S or Si, or R¹ and R² together form an optionally substituted hydrocarbon bridge or an optionally substituted α,ω-dioxahydrocarbon bridge between X and Y;
R³ and R⁴ are independently selected from the group consisting of hydrogen, halogen, optionally substituted alkyl, optionally substituted aryl, optionally substituted alkyl-Z′— and optionally substituted aryl-Z′—, where Z′ is N, O, S or Si, or R³ and R⁴ together form an optionally substituted hydrocarbon bridge or an optionally substituted α,ω-dioxahydrocarbon bridge between the two carbon atoms to which they are attached;
R⁵ and R⁶ are independently selected from the group consisting of hydrogen, optionally substituted alkyl and optionally substituted aryl, or R⁵ and R⁶ together with the nitrogen atom to which they are attached form an optionally substituted azacycloalkyl group;
or an enantiomer or diastereomer thereof or a salt, optionally a pharmaceutically acceptable salt, of any of these,

wherein if either X or Y or both is O, [symbol] is a single bond and if X═O, R² is absent and if Y═O, R¹ is absent.

3-Deazaneplanocin A may be excluded from the scope of this aspect. Any one or more, optionally all, of the following compounds may be excluded from the scope of this aspect: aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (1R,2S,3R)-3-(6-amino-9H-purin-9-yl)-1,2-cyclopentanediol hydrochloride, 2′,3′-O-isopropylidene-3-deazaneplanocin A, (1S,2R,5R)-5-(6-amino-9H-purin-9-yl)-3-methylcyclopent-3-ene-1,2-diol hydrochloride. 3-Deazaneplanocin A, aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (±)-(1R,2S,3R)-3-(6-amino-9H-purin-9-yl)-1,2-cyclopentanediol hydrochloride, 2′,3′-O-isopropylidene-3-deazaneplanocin A and (1S,2R,5R)-5-(6-amino-9H-purin-9-yl)-3-methylcyclopent-3-ene-1,2-diol hydrochloride may all be excluded from the scope of this aspect.

The following options may be used in conjunction with the first aspect, either individually or in any suitable combination.

The compound may be such that:
X and Y are both C;
R¹ and R² are independently hydrogen, halogen, an aliphatic, arylaliphatic or hydrocarbyl group having between 1 and 8 main chain carbon atoms and between 0 and 3 heteroatoms, each heteroatom, independently, being N, O, S, Si (wherein if the heteroatom is N or Si, the other group(s) attached thereto are, independently, hydrogen, aryl or aliphatic);
R³ and R⁴ are independently hydroxy, alkoxy, cycloalkyloxy, aryloxy, arylalkoxy or arylcycloaalkyloxy groups comprising between 0 and 3 heteroatoms, each heteroatom, independently, being N, O, S, or Si (wherein if the heteroatom is N or Si, the other group(s) attached thereto are, independently, hydrogen, aryl or aliphatic) or R³ and R⁴ are linked so as to define an α,ω-dioxahydrocarbon bridge between the two carbon atoms to which they are attached; and
R⁵ and R⁶ are independently hydrogen, aliphatic, cycloaliphatic, aromatic, arylaliphatic or arylcycloaliphatic hydrocarbyl groups comprising between 0 and 3 heteroatoms, each heteroatom, independently, being N, O, S, or Si (wherein if the heteroatom is N or Si, the other group(s) attached thereto are, independently, hydrogen, aryl or aliphatic).

The compound may be such that:
X and Y are both C;
R¹ and R² are independently hydrogen or halogen or aliphatic, arylaliphatic, hydrocarbyl group comprising 1-8 main chain carbon atoms and 0-3 heteroatoms being N, O, S, Si (wherein if the heteroatom is N or Si, the other group(s) attached thereto are, independently, hydrogen, aryl or aliphatic), or a halogen such as Cl or F;
R³ and R⁴ are independently hydrogen or halogen or carbon or aliphatic, cylcoaliphatic, aromatic, arylcycloaliphatic or arylaliphatic hydrocarbyl group or may be linked so as to define an aliphatic hydrocarbyl bridge;
R⁵ and R⁶ are, independently hydrogen or aliphatic, cycloaliphatic, aromatic, arylaliphatic, or arylcycloaliphatic hydrocarbyl groups, that comprise 0-3 heteroatoms being N, O, S, or Si (wherein if the heteroatom is N or Si, the other group(s) attached thereto are, independently, hydrogen, aryl or aliphatic).

X and Y may both be C.

R¹ may be H.

In some embodiments either X or Y is O and the other is C. The compound may be such that X═C, Y═O and [symbol] is a single bond, whereby R¹ is absent.

R³ and R⁴ may both be OH or they may together form a protected vicinal diol. R³ and R⁴ may together form an —OC(Me₂)O— group.

The compound may be ((3R,4S,5R)-3-(6-amino-9H-purin-9-yl)-4,5-dihydroxycyclopent-1-enyl)methyl benzoate hydrochloride.

The compound may show activity to activate E2F1-induced apoptosis of at least about 15%. It may show activity to activate E2F1-induced apoptosis in the presence of 4-OHT of at least about 25%. It may show apoptosis induction in colon cancer cells with histone deacetylase inhibitor TSA of at least about 40%. It may be capable of inhibiting the function of Polycomb repressive complex 2 (PRC2) proteins.

In an embodiment of the invention there is provided a compound of structure I wherein:

X and Y are both C;

### A is C or N;

[symbol] is a single bond or a double bond;

### R¹ is H;

R² is selected from the group consisting of hydrogen and optionally substituted alkyl;
R³ and R⁴ either both are OH or together they form a protected vicinal diol, e.g. an —OC(Me₂)O— group;
R⁵ and R⁶ are both H;
or an enantiomer or diastereomer thereof or a salt (e.g. a pharmaceutically acceptable salt) of any of these.

In a second aspect of the invention there is provided use of a compound according to the first aspect for the manufacture of a medicament for the treatment of cancer. The cancer may be a cancer characterized by overexpression of EZH2 (enhancer of zeste homolog 2). This gene encodes a member of the Polycomb-group family (PcG), which form multimeric protein complexes. These contribute to maintenance of the transcriptional repressive state of genes over successive cell generations. Cancers that may be treated by the medicament include breast cancer and prostate cancer (particularly metastatic prostate cancer). The compound may be aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (1R,2S,3R)-3-(6-amino-9H-purin-9-yl)-1,2-cyclopentanediol hydrochloride, 2′,3′-O-isopropylidene-3-deazaneplanocin A or (1S,2R,5R)-5-(6-amino-9H-purin-9-yl)-3-methylcyclopent-3-ene-1,2-diol hydrochloride or an enantiomer or diastereomer thereof or a salt (e.g. a pharmaceutically acceptable salt) of any of these.

In a third aspect of the invention there is provided use of a compound according to the first aspect in therapy. In particular there is provided use of a compound according to the first aspect for the treatment of cancer, e.g. breast cancer and prostate cancer (particularly metastatic prostate cancer). For use in treatment of cancer, the compound may be aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (1R,2S,3R)-3-(6-amino-9H-purin-9-yl)-1,2-cyclopentanediol hydrochloride, 2′,3′-O-isopropylidene-3-deazaneplanocin A or (1S,2R,5R)-5-(6-amino-9H-purin-9-yl)-3-methylcyclopent-3-ene-1,2-diol hydrochloride or an enantiomer or diastereomer thereof or a salt (e.g. a pharmaceutically acceptable salt) of any of these.

In a fourth aspect of the invention there is provided a composition, in particular a pharmaceutical composition, comprising a compound according to the first aspect, or an enantiomer, diastereomer or pharmaceutically acceptable salt thereof, together with one or more pharmaceutically acceptable carriers, diluents, excipients or adjuvants. The composition may be suitable for the treatment of cancer, e.g. breast cancer and prostate cancer (particularly metastatic prostate cancer). If the composition is suitable for the treatment of cancer, the compound may be aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (1R,2S,3R)-3-(6-amino-9H-purin-9-yl)-1,2-cyclopentanediol hydrochloride, 2′,3′-O-isopropylidene-3-deazaneplanocin A or (1S,2R,5R)-5-(6-amino-9H-purin-9-yl)-3-methylcyclopent-3-ene-1,2-diol hydrochloride or an enantiomer or diastereomer thereof or a salt (e.g. a pharmaceutically acceptable salt) of any of these.

In a fifth aspect of the invention there is provided a method of treating cancer, e.g. breast cancer and prostate cancer (particularly metastatic prostate cancer), comprising administering to a patient in need thereof a clinically effective amount of a compound according to the first aspect, or an enantiomer, diastereomer or pharmaceutically acceptable salt thereof, or of a composition according to the fourth aspect. The compound may be aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (1R,2S,3R)-3-(6-amino-9H-purin-9-yl)-1,2-cyclopentanediol hydrochloride, 2′,3′-O-isopropylidene-3-deazaneplanocin A or (1S,2R,5R)-5-(6-amino-9H-purin-9-yl)-3-methylcyclopent-3-ene-1,2-diol hydrochloride or an enantiomer or diastereomer thereof or a salt (e.g. a pharmaceutically acceptable salt) of any of these.

### BRIEF DESCRIPTION OF THE DRAWINGS

A preferred embodiment of the present invention will now be described, by way of an example only, with reference to the accompanying drawings wherein:

FIG. 1 is a bar chart showing % apoptosis with and without 4-OHT for various compounds;

FIG. 2 shows body weight changes for a control group and a group administered compound D3;

FIG. 3 shows tumour volume changes for the control group the group administered compound D3;

FIG. 4 shows % growth inhibition for the group administered compound D3;

FIG. 5 shows the tumour volume changes on administration of compound 13;

FIG. 6 shows body weight change for the group administered compound 13;

FIG. 7 shows tumour volume growth inhibition for the group administered compound 13.

### DETAILED DESCRIPTION OF THE PREFERRED EMBODIMENTS

In this specification, the numbering of the atoms in the compounds described is as shown below:

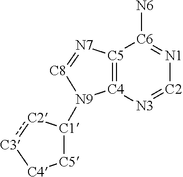

In the event that one of the atoms in the above structure is replaced by a different atom (e.g. if N3 is replaced by a carbon atom) this may be referred to as C3, or may be referred to as being in position 3. Where a particular substituent is not explicitly described or shown, it will in general be hydrogen unless the context indicates otherwise.

The invention relates to compounds of general structure I and to enantiomers or diastereomers thereof and to salts of any of these.

X and Y in structure I are independently C or O. Commonly they are both C. In particular, in the event that the X—Y bond is a double bond, the substituent on C2′ (i.e. X when X is C) may be H, or in the event that the X—Y bond is a single bond, the substituents on C2′ may both be H. In the event that the X—Y bond is a single bond, either of the substituents on C2′ (e.g. R¹) may be oriented up and the other down and either of the substituents on C3′ (e.g. R²) may be oriented up and the other down. In some instances either X or Y (or both) is O. In particular, one may be C and the other is O. In a particular instance, X is C and Y is O. In such instances the bond between them is a single bond, and there will be no substituent on which ever of X and Y is O.

A may be C or N. In the event that A is C this represents the same ring structure as 3-deazaneplanacin A (when X and Y are both C and are joined by a double bond). If A is C, the substituent on it may be H, or may be some other substituent such as an alkyl group or an aryl group (as defined below).

Alkyl groups described herein may be C1 to C12 alkyl groups, or C1 to C8, C1 to C6 or C1 to C4. They may be for example methyl, ethyl, propyl, isopropyl, butyl (m, s or t) etc. They may be linear, or they may (except for C1 and C2) be branched or cyclic alkyl. They may optionally contain one or more double or triple bonds (i.e. they may be alkenyl and/or alkynyl). They may optionally be substituted with one or more substituents. Each substituent on the alkyl groups may, independently, be R—B— (where R is hydrogen or an alkyl group as described above or an aryl group as described below, both being optionally substituted and B is O, S, N or Si) or halogen (e.g. F, Cl, Br or I). In the event that B is N or Si, the other (i.e. hitherto undefined) position(s) on B may (each independently) have an alkyl or aryl group as described herein. The alkyl groups may be arylalkyl groups. They may be arylcycloalkyl groups. They may represent alkoxyalkyl or aryloxyalkyl or alkylaminoalkyl (e.g. mono- or dialkylaminoalkyl) groups or arylaminoalkyl groups or alkanethioalkyl groups or arylthioalkyl groups or alkylsilylalkyl (e.g. trialkylsilylalkyl) groups or arylsilylalkyl groups (e.g. trialkyl-, aryldialkyl- or diarylalkyl-silylalkyl groups). They may represent oligoether groups (e.g. H(CH₂CH₂O)ₙCH₂CH₂—) or oligoaminogroups (e.g. H(CH₂CH₂NH)ₙCH₂CH₂—) where n=1 to about 6. The total number of atoms (other than hydrogen but including heteroatoms) in the main chain of the alkyl group may be 3 to 20, or 3 to 12 or 3 to 8.

Aryl groups described may be monocyclic aromatic groups or they may be bicyclic, tricyclic or oligocyclic. They may (except for monocyclic instances) be fused ring aromatic groups. They may be carbocyclic or they may be heterocyclic. They may for example be phenyl, naphthyl, anthracyl, pyridyl, furyl, pyrrolyl, thiofuryl, imidazolyl, indolyl, quinolinyl, naphthyridyl etc. They may optionally be substituted with one or more substituents. Each substituent on the aryl groups may, independently, be R—B—, where R and B are as described above (under “alkyl groups”). They may be for example alkylaryl groups or di-, tri-, tetra- or penta-alkylaryl groups, or may be alkoxyaryl groups or alkoxyalkoxyaryl groups. They may be haloaryl groups.

R¹ and R² may be hydrogen, halogen, optionally substituted alkyl, optionally substituted aryl, optionally substituted alkyl-Z— or optionally substituted aryl-Z—, where Z is N, O, S or Si. In the event that Z is N or Si, the other (i.e. hitherto undefined) position(s) on Z may (each independently) have hydrogen, an alkyl group or an aryl group as described above. R¹ and R² may together form an optionally substituted hydrocarbon bridge or an optionally substituted α,ω-dioxahydrocarbon bridge between X and Y. The substituents may be alkyl, aryl, R—B— or halogen, as described above. The hydrocarbon bridge may have formula —(CH₂)ₙ—, where n is an integer. n may be between 1 and 6, or 2 and 6, 3 and 6, 4 and 6 or 3 and 5, e.g. 1, 2, 3, 4, 5 or 6. In some instances the bridge may have substituents as described above. The substituents themselves may form a ring, whereby the substituent on N9 of the ring system is a fused tricyclic ring system. In many embodiments, R¹ is hydrogen, and in some embodiments both R¹ and R² are both hydrogen. In some embodiments R² is an alkyl group having an oxygen substituent (e.g. hydroxyl, alkoxy or aryloxy).

R³ and R⁴ may, independently, be hydrogen, halogen (e.g. chloro, bromo, iodo or fluoro), optionally substituted alkyl, optionally substituted aryl, optionally substituted alkyl-Z′— or optionally substituted aryl-Z′—, where Z′ is N, O, S or Si. In the event that Z′ is N or Si, the other (i.e. hitherto undefined) position(s) on Z′ may (each independently) have hydrogen, an alkyl group or an aryl group as described above. R³ and R⁴ may together form an optionally substituted hydrocarbon bridge or an optionally substituted α,ω-dioxahydrocarbon bridge between the two carbon atoms to which they are attached. Broadly the choice of options for R³ and R⁴ is the same as for R¹ and R² above. In some embodiments, R³ and R⁴ are both alkoxy, aryloxy or together form an α,ω-dioxahydrocarbon bridge. Suitable bridges include typical protecting groups for vicinal diols, for example methylene acetal, eththylidene acetal or isopropylidene acetal (acetonide: —OC(Me₂)O—).

R⁵ and R⁶ may be hydrogen, optionally substituted alkyl or optionally substituted aryl. R⁵ and R⁶ may, together with the nitrogen atom to which they are attached, form an optionally substituted azacycloalkyl group. The ring of the azacycloalkyl group may have about 3 to about ring members, or 4 to 8, 5 to 8 or 5 to 7 members. In many embodiments R⁵ and R⁶ are both hydrogen whereby N6 represents a primary amino group. In other embodiments N6 represents a secondary or tertiary amino group. Broadly the choice of options for R⁵ and R⁶ is the same as for R¹ and R² above with the exception that they may not be halogens or form a α,ω-dioxahydrocarbon bridge.

The present invention also encompasses enantiomers and diastereomers of the compounds described above. It also encompasses solvates, e.g. hydrates, of the compounds and of their enantiomers and diastereomers. It also encompasses salts of the compounds and of their enantiomers and diastereomers. The salts may be clinically acceptable salts. They may pharmaceutically acceptable. They may be for example chlorides, bromides, sulfates, phosphates or some other suitable salt.

The present invention excludes from its scope hitherto known compounds, including 3-deazaneplanocin A or aristeromycin.

The compound may show activity to activate E2F1-induced apoptosis of at least about 15%, or at least about 20 or 25%, or about 15 to 25%, 15 to 30%, 15 to 20% or 20 to 25%. In this context, transcription factor E2F1, which is involved in the action of tumour suppressing proteins, was engineered with ER receptor ligand binding domain. (ER receptor is a nuclear hormone type of intracellular oestrogen receptor.) The compound may show activity to activate E2F1-induced apoptosis in the presence of 4-OHT of at least about 25% or of at least about 30, 40, 50, 60, 70 or 80%, or of about 25 to about 80%, or about 30 to 80, 50 to 80, 60 to 80 or 50 to 70%, e.g. about 25, 30, 35, 40, 45, 50, 55, 60, 65, 70, 75, 80 or 85%. 4-OHT is 4-hydroxytamoxifen, an anti-estrogenic metabolite of tamoxifen with a much higher affinity for oestrogen receptors than tamoxifen itself. The compound may show apoptosis induction in colon cancer cells with histone deacetylase inhibitor TSA (Trichostatin A) of at least about 40%, or at least about 50, 60, 70 or 80%, or about 40 to about 90%, or about 50 to 90, 70 to 90, 40 to 60 or 50 to 80%, e.g. about 40, 50, 60, 70, 80 or 90%. It may be capable of inhibiting the function of Polycomb repressive complex 2 (PRC2) proteins. In this context, activity % refers to the percentage of cell undergoing apoptosis (death) under the combination drug treatment of DZNep analogue with TSA for 48 h.

The invention additionally provides for therapeutic uses of the compound, in particular for the treatment of various cancers, as well as for the preparation of medicaments and compositions for such uses. The patient in such applications may be human or may be non-human. The patient may be a non-human mammal or a bird. The patient may be a primate, e.g. a non-human primate. It may be a domestic animal. It may be a farm animal. It may be a wild animal. The invention also encompasses non-therapeutic uses of the compounds of the invention.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

These compositions can be administered by standard routes. In general, the compositions may be administered by the parenteral (e.g., intravenous, intraspinal, subcutaneous or intramuscular), oral or topical route. More preferably administration is by the parenteral route.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emulsifiers, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

The present invention therefore relates to 3-deazaneplanocin A (DZNep) derivatives and/or analogues. Suitable therapeutically attractive examples target histone methylation and PRC2 complex, and may therefore be useful for cancer therapy. The present specification describes the chemical synthesis and biological testing of potentially biologically active compounds.

The objectives of the work were:
i) to develop a library of compounds based on the 3-deazaneplanocin A (DZNep) core structure to inhibit the Polycomb repressive complex 2 (PRC2) proteins, and
ii) to screen the biological activity of these compounds.

The present invention broadly relates therefore to compounds based on the 3-deazaneplanocin A (DZNep) core structure (Structure 1 and 2) and their respective biological activities.

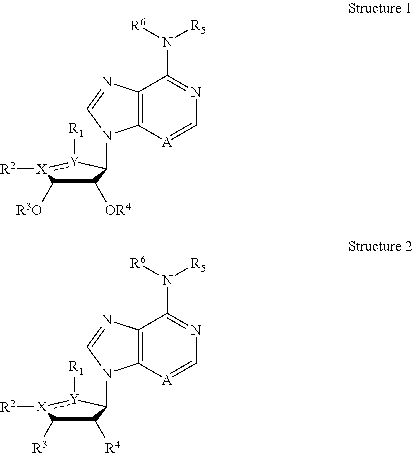

With respect to structure 1:
A may be carbon or nitrogen;
X and Y may be carbon;
the bond between X and Y may be saturated or unsaturated;
R¹ and R² may independently be hydrogen or halogen (e.g. Cl, F) or aliphatic, arylaliphatic, hydrocarbyl group comprising 1 to 8 main chain carbon atoms and 0 to 3 heteroatoms, each heteroatom, independently being N, O, S, Si (if the heteroatom is N or Si, the other group(s) attached thereto may, independently, be hydrogen, aryl or aliphatic);
R³, R⁴, R⁵ and R⁶ may independently be hydrogen or an aliphatic, cycloaliphatic, aromatic, arylaliphatic or arylcycloaliphatic hydrocarbyl group comprising 0 to 3 heteroatoms, each heteroatom, independently, being N, O, S, or Si (if the heteroatom is N or Si, the other group(s) attached thereto may, independently, be hydrogen, aryl or aliphatic), where R³ and R⁴ may optionally be linked so as to define an aliphatic hydrocarbyl bridge.
With respect to structure 2:
A may be carbon or nitrogen;
X and Y may be carbon;
the bond between X and Y may be saturated or unsaturated;
R¹ and R² may, independently, be hydrogen or halogen or aliphatic, arylaliphatic, hydrocarbyl group comprising 1 to 8 main chain carbon atoms and 0 to 3 heteroatoms, each heteroatom independently being N, O, S, Si (if the heteroatom is N or Si, the other group(s) attached thereto may, independently, be hydrogen, aryl or aliphatic);
R³ and R⁴ may, independently, be hydrogen or halogen or an aliphatic, cylcoaliphatic, aromatic, arylcycloaliphatic or arylaliphatic hydrocarbyl group or may be linked so as to define an aliphatic hydrocarbyl bridge;
R⁵ and R⁶ may, independently, be hydrogen or an aliphatic, cycloaliphatic, aromatic, arylaliphatic, or arylcycloaliphatic hydrocarbyl group comprising 0 to 3 heteroatoms, each heteroatom, independently, being N, O, S, or Si(if the heteroatom is N or Si, the other group(s) attached thereto may, independently, be hydrogen, aryl or aliphatic).

A library of 21 compounds with different heterocyclic ring based on the lead compound, 3-deazaneplanocin A (DZNep), was synthesized and purified. In addition, modification of the cyclopentene ring and side-arm, i.e. the group attached to C3 of the cyclopentene (or cyclopentane) ring was also investigated. Results of biological testing are summarized in Table 1.

### Apoptotic Assays for Structure-Activity Relationship Analysis

Two assays were used to measure the compound's activity in induce apoptosis. The first assay measures the activity of compound to activate E2F1-induced apoptosis. In this assay, transcription factor E2F1 was engineered with ER receptor ligand binding domain. Addition of 4-OHT will be able to activate ER-E2F1 complex activity. DZNep has been found to induce E2F1-induced apoptosis so this assay was used to compare the new derived compounds with DZNep for their ability to induce apoptosis in this cellular system.

The second assay was designed to measure synergistic effect of new compounds with histone deacetylase inhibitor TSA for apoptosis induction in colon cancer cells. DZNep is known to synergy with TSA to induce strong apoptosis in colon cancer cells ((Jiang et al., Cancer Cell, 13, 529-541, 2008). Again the new compounds are compared with DZNep in the context of inducing apoptosis with TSA. Assays were conducted in accordance with Jiang et al (above).

TABLE 1

| | | | E2F1 | Combination with |
|---|---|---|---|---|
| | | | Induced apoptosis | HDAC inhibitor |
| | | | (+4-OHT) | TSA |
| | Structure | Synthesis | % Apoptosis | % Apoptosis |
| 1 | 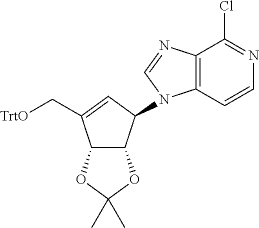 | a | No activity | 20% |
| 2 | 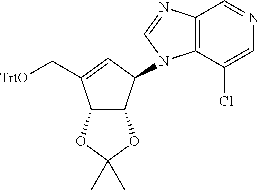 | a | No activity | 40% |
| 3 | 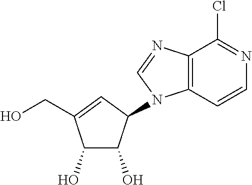 | b | No activity | No activity |
| 4 | 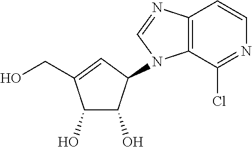 | b | No activity | No activity |
| 5 | 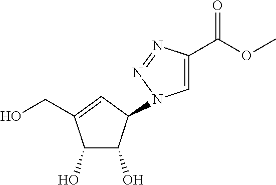 | a | No activity | No activity |
| 6 | 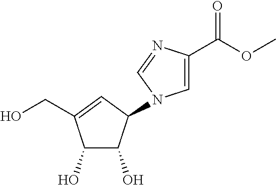 | a | No activity | No activity |
| 7 | 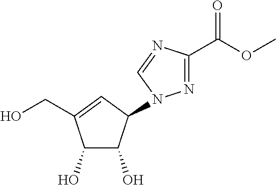 | a | No activity | No activity |
| 8 | 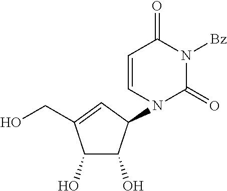 | a | No activity | No activity |
| 9 | 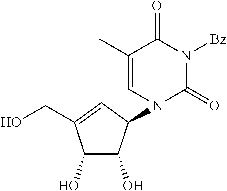 | a | No activity | No activity |
| 10 | 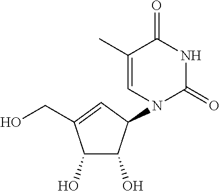 | a | No activity | No activity |
| 11 | 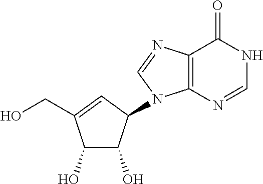 | a | No activity | No activity |
| 12 | 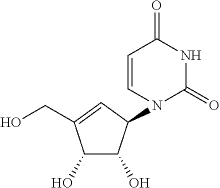 | a | No activity | No activity |
| 13 | 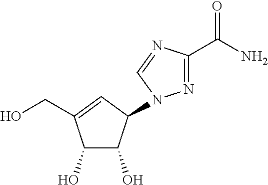 | a | No activity | No activity |
| 14 | 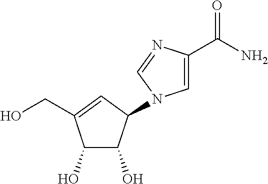 | a | No activity | No activity |
| 15 | 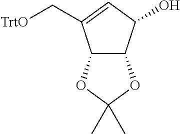 | a | No activity | No activity |
| 16 | 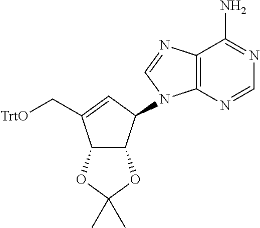 | a | No activity | No activity |
| 17 | 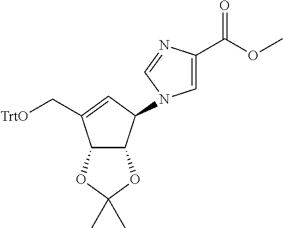 | a | No activity | No activity |
| 18 | 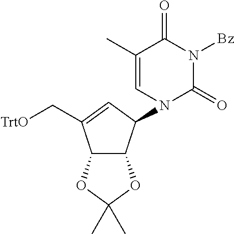 | a | No activity | No activity |
| 19 | 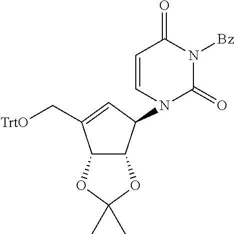 | a | No activity | No activity |
| 20 | 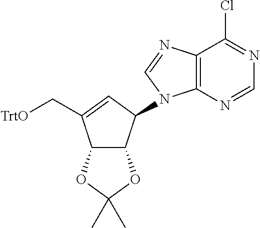 | a | No activity | No activity |
| 21 | 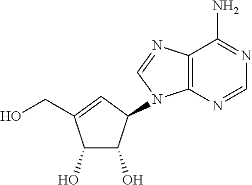 | c | ND | 65% |
| 22 | 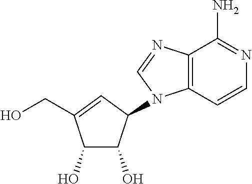 | Lead compound | 25% (65%) | 70-75% |
| 23 | 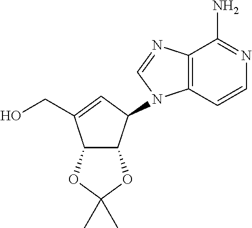 | Example 1 | 18% (25%) | No activity |
| 24 | 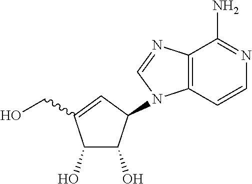 | Example 2 | 28% (60%) | 70% |
| 25 | 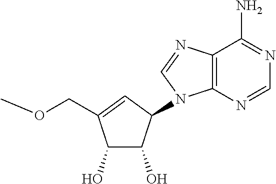 | Example 5 | 25% (60%) | 82% |
| 26 | 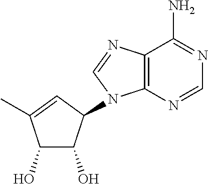 | Example 7 | 25% (40%) | 40% |
| 27 | 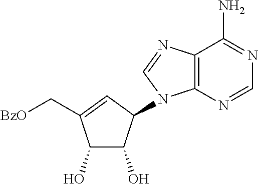 | Example 9 | 15% (30%) | 51% |
| 28 | 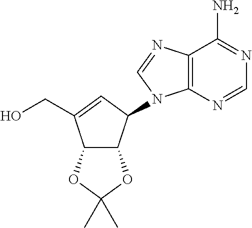 | d | No activity | No activity |
| 29 | 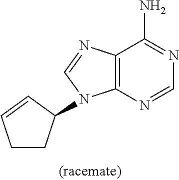 | Example 10 | No activity | No activity |
| 30 | 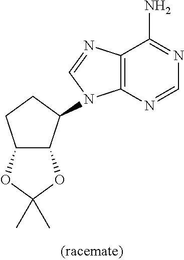 | Example 11 | No activity | No activity |
| 31 | 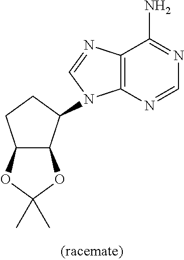 | Example 11 | No activity | No activity |
| 32 | 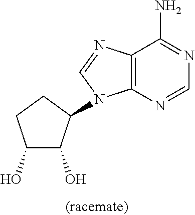 | Example 12 | 21% (44%) | 58% |
| 33 | 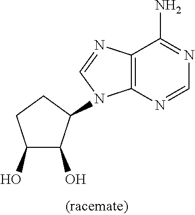 | Example 13 | No activity | No activity |

a) Cho, J. H., Bernard, D. L., Sidwell, R. W., Kern, E. R., Chu, C. K. Synthesis of Cyclopentenyl Carbocyclic Nucleosides as Potential Antiviral Agents Against Orthopoxviruses and SARS J. Med. Chem., 49, 1140-1148 (2006)
b) Yang, M., Zhou, J., Schneller, S. W. The Mitsunobu reaction in preparing 3-deazapurine carbocyclic nucleosides. Tetrahedron 63, 1295-1300 (2006).
c) Michel, B. Y., Strazewski, P. Synthesis of (−)-neplanocin A with the highest overall yield via an Efficient Mitsunobu coupling. Tetrahedron 63, 9836-9841 (2007).
d) U.S. Pat. No. 4,613,666.

### Example 1

### 2′,3′-O-Isopropylidene-3-deazaneplanocin A

A mixture of 3-deazaneplanocin A hydrochloride (DZnep) (20 mg, 0.067 mmol), 0.5 mL of DMF and 1 mL of 1 M HCl in diethyl ether in 5 mL of acetone was stirred at room temperature for 18 h and then neutralized with triethylamine (TEA). The solvent was removed under reduced pressure. The residue was purified by flash chromatography (silica gel, MeOH/TEA/DCM=10:10:80) to afford 18 mg (89%) of the title compound. ¹H NMR (MeOD, 400 MHz): δ 8.175 (s, 1H), 7.68 (d, J=6.4 Hz, 1H), 7.12 (d, J=6.4 Hz, 1H), 5.55 (s, 1H), 5.36 (d, J=6.0 Hz, 1H), 4.68 (d, J=6.0 Hz, 1H), 4.365 (s, 2H), 1.48 (s, 3H), 1.35 (s, 3H); ESI MS m/z for C₁₅H₁₈N₄O₃ calculated: 302.14. found: 303.13 (M+H)⁺.

### Example 2

### 3-Deazaaristeromycin hydrochloride (D2)

To a solution of 3-deazaneplanocin A hydrochloride (DZnep) (15 mg, 0.05 mmol) in 2 mL of MeOH was added 10 mg of 10% palladium on charcoal. The suspension was stirred for 18 h at room temperature under a hydrogen atmosphere. The mixture was filtered with a pad of celite to remove the palladium. The product was purified with preparative LCMS in 50% yield (ratio of two diastereoisomers=1:1). ESI MS m/z for C₁₂H₁₆N₄O₃ calculated: 264.12. found: 265.11 (M+H)⁺.

### Example 3

### (1R,4R,5S)-9-N-[3-(hydroxymethyl)-4,5-O,O-isopropylidene-2-cyclopenten-L-yl]-N⁶,N⁶-bis-(tert-butoxycarbonyl)adenine

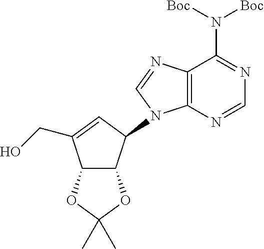

To a solution of (1R,4R,5S)-9-N-[3-(trityloxymethyl)-4,5-O,O-isopropylidene-2-cyclopenten-L-yl]-N⁶,N⁶-bis-(tert-butoxycarbonyl)adenine [Tetrahedron lett. 2006, (47) 9187-9189.] (225 mg, 0.45 mmol) in 20 mL of acetone was added 2,2-dimethoxypropane (20 mL) and p-toluenesulfonic acid monohydrate (42.8 mg, 0.225 mmol) at room temperature. The acidic solution was allowed to stir for 18 h at room temperature. The reaction mixture was quenched with 300 mg of solid sodium bicarbonate. The solvent was evaporated in vacuo and the residue was added water (20 mL) and DCM (20 mL). Separated the two phase. The aqueous layer was extracted by DCM (3×20 mL). The combined organic layers were dried with MgSO₄ and concentrated in vacuo. The residue purified by flash chromatography on silica gel (petroleum ether/EtOAc=2:1 to 1:2) to give the title compound in 175 mg (75%). ¹H NMR (400 MHz, CDCl₃) δ 8.87 (s, 1H,), 7.99 (s, 1H), 5.81 (bs, 1H), 5.65 (bs, 1H), 5.41 (d, 1H, J=5.1 Hz), 4.75 (d, 1H, J=5.1 Hz,), 4.47 (dt, 2H, J=15.4, 2.2 Hz), 1.49 (s, 3H), 1.45 (s, 18H), 1.36 (s, 3H); HR-MS (ESI⁻) m/z for C₂₄H₃₂N₅O₇ calculated 502.2307. found 502.2299 (M−H)⁻.

### Example 4

### (1R,4R,5S)-9-N-[3-(methoxymethyl)-4,5-O,O-isopropylidene-2-cyclopenten-L-yl]-N⁶,N⁶-bis-(tert-butoxycarbonyl)adenine

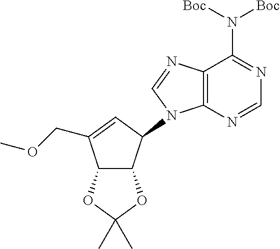

To a stirred suspension of sodium hydride (60% w/w, 27 mg, 0.675 mmol) in 20 mL of dry DMF was added dropwise a solution of (1R,4R,5S)-9-N[3-(hydroxymethyl)-4,5-O,O-isopropylidene-2-cyclopenten-L-yl]-N⁶,N⁶-bis-(tert-butoxycarbonypadenine (320 mg, 0.62 mmol) in 5 ml of dry DMF at 0° C. under argon. The resulting yellow mixture was stirred at 0° C. for 20 min and warmed to room temperature for 1 h. The reaction mixture was cool back to 0° C. and 5 mL of dry DMF solution of methyl iodide (180 mg, 1.2 mmol) was added. After 1 h at room temperature, the reaction was cooled to 0° C. and quenched with of 5 mL of saturated ammonium chloride solution. The aqueous layer was extracted by diethyl ether (3×20 mL). The combined organic layers were washed with water (20 mL), dried with MgSO₄ and concentrated in vacuo. The residue was purified by flash chromatography on silica gel (diethyl ether/pentane=1:10 to 2:1) to give the title compound in 160 mg (54%). ¹H NMR (400 MHz, CDCl₃) δ 8.77 (s, 1H,), 7.98 (s, 1H), 5.83 (bs, 1H), 5.66 (bs, 1H), 5.40 (d, 1H, J=5.2 Hz), 4.83 (dd, 2H, J=28.0 and 14.4 Hz,) 4.70 (d, 1H, J=5.6 Hz,), 3.49 (s, 3H), 1.48 (bs, 21H), 1.35 (s, 3H);

### Example 5

### (1S,2R,5R)-5-(6-amino-9H-purin-9-yl)-3-(methoxymethyl)cyclopent-3-ene-1,2-diol hydrochloride (D3)

To a solution of (1R,4R,5S)-9-N-[3-(methoxymethyl)-4,5-O,O-isopropylidene-2-cyclopenten-L-yl]-N⁶,N⁶-bis-(tert-butoxycarbonypadenine (16.7 mg, 0.032 mmol) in 1 mL of MeOH was added 1 mL of 1 M HCl in diethyl ether. The mixture was stirred at room temperature for 18 h. The solvent was removed under reduced pressure. The residue was washed with DCM to give 8 mg of the title compound (80%). ¹H NMR (MeOD, 400 MHz): δ 8.33 (s, 1H), 8.23 (s, 1H), 5.90 (s, 1H), 5.49 (s, 1H), 4.62 (d, J=5.6 Hz, 1H), 4.37 (t, J=6.0 Hz, 1H), 4.32 (s, 2H), 3.31 (s, 3H); HR-MS (ESI⁺) m/z for C₁₂H₁₆N₅O₃ calculated 278.1248. found 278.1234 (M+H)⁺. for C₁₂H₁₅N₅NaO₃ calculated 300.1067. found 300.1053 (M+Na)⁺.

### Example 6

### 9-((3aS,4R,6aR)-2,2,6-trimethyl-4,6a-dihydro-3aH-cyclopenta[d][1,3]dioxol-4-yl)-9H-purin-6-amine

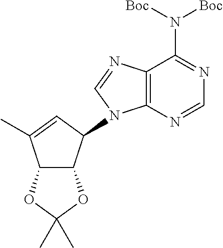

To a solution of (1R,4R,5S)-9-N-[3-(hydroxymethyl)-4,5-O,O-isopropylidene-2-cyclopenten-L-yl]-N⁶,N⁶-bis-(tert-butoxycarbonypadenine (26 mg, 0.05 mmol) in 2 mL of DCM was added thiocarbonyldiimdazole (15 mg, 0.075 mmol). The reaction mixture was stirred at ambient temperature for 18 h and evaporated in vacuo. The residue was dissolved in toluene. Bu₃SnH (44 mg, 0.15 mmol) and a catalytic amount of AIBN (1 mg) was added to the solution and heated to reflux for 8 h. The reaction was cool to room temperature. The mixture was evaporated in vacuo and the residue was purified by flash chromatography on silica gel (MeOH/DCM=0:100 to 10:90) to give the title compound in 68% yield (9.8 mg).

### Example 7

### (1S,2R,5R)-5-(6-amino-9H-purin-9-yl)-3-methylcyclopent-3-ene-1,2-diol hydrochloride

The same experimental procedure employed in the example 5. Compound 9-((3aS,4R,6aR)-2,2,6-trimethyl-4,6a-dihydro-3 aH-cyclopenta[d][1,3]dioxol-4-yl)-9H-purin-6-amine (9.8 mg, 0.02 mmol) was hydrolysis to give 5.2 mg (92%) of the title compound. ¹H NMR (MeOD, 400 MHz): δ 8.34 (s, 1H), 8.27 (s, 1H), 5.66 (s, 1H), 5.54 (s, 1H), 4.48 (m, 1H), 4.32 (m, 1H), 1.90 (s, 3H); HR-MS (ESI⁺) m/z for C₁₁H₁₃N₅NaO₂, calculated 270.0962. found 270.0966 (M+Na)⁺.

### Example 8

### (1R,4R,5S)-9-N-[3-(benzoyloxymethyl)-4,5-O,O-isopropylidene-2-cyclopenten-L-yl]-N⁶,N⁶-bis-(tert-butoxycarbonyl)adenine

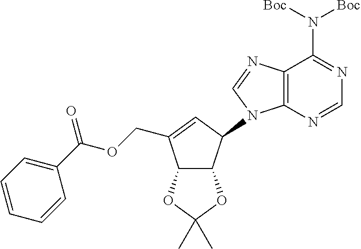

To a solution of (1R,4R,5S)-9-N[3-(hydroxymethyl)-4,5-O,O-isopropylidene-2-cyclopenten-L-yl]-N⁶,N⁶-bis-(tert-butoxycarbonyl)adenine (10 mg, 0.02 mmol) in 5 mL of DCM was added 0.1 mL of TEA, 1 mg of DMAP and 2.5 μL of benzoyl chloride (0.022 mmol). The resultant mixture was stirred at room temperature under an argon atmosphere for 18 h. After concentrated the solvent in vacuo, the product was purified under flash chromatography on silica gel (petroleum ether/diethyl ether=1:1). The product was obtained in 12 mg (98%) as a white solid.

### Example 9

### ((3R,4S,5R)-3-(6-amino-9H-purin-9-yl)-4,5-dihydroxycyclopent-1-enyl)methyl benzoate hydrochloride

The same experimental procedure used in the example 5. A 10 mg (0.016 mmol) of (1R,4R,5S)-9-N-[3-(benzoyloxymethyl)-4,5-O,O-isopropylidene-2-cyclopenten-L-yl]-N⁶,N⁶-bis-(tert-butoxycarbonyl)adenine gave 5 mg (78%) of the title product. ¹H NMR (MeOD, 400 MHz): δ 8.34 (s, 2H), 8.07 (d, J=7.6 Hz, 2H), 7.62 (t, J=7.2 Hz, 1H), 7.49 (t, J=7.6 Hz, 2H), 6.065 (s, 1H), 5.64 (s, 1H), 5.09 (s, 2H), 4.76 (d, J=5.2 Hz, 1H), 4.45 (t, J=5.2 Hz, 1H); HR-MS (ESI⁺) m/z for C₁₈H₁₈N₅O₄ calculated 368.1353. found 368.1336 (M+H)⁺. for C₁₈H₁₇N₅NaO₄ calculated 390.1173. found 390.1155 (M+Na)⁺.

### Example 10

### (±)-9-(cyclopent-2-enyl)-9H-purin-6-amine

To a solution of cyclopetenol (84 mg, 1 mmol), adenine (202 mg, 1.5 mmol) and Ph₃P (524 mg, 2 mmol) in 2.0 mL of anhydrous THF was added diisopropyl azodicarboxylate (DIAD, 393 μL, 2 mmol) at 0° C. under an argon atmosphere, and the mixture was allowed to stir for 18 h at room temperature. The solvent was removed under reduced pressure. The residue was purified by flash chromatography (silica gel, MeOH/DCM=10:90) to give 120 mg (60%) of corresponding compound. ¹H NMR (CDCl₃, 400 MHz): 8.32 (s, 1H), 7.73 (s, 1H), 6.59 (s, 1H), 6.25 (2d, J=2.0 Hz, 5.6 Hz, 1H), 5.86 (dd, J=2.4, 5.6 Hz, 1H), 5.69 (m, 1H), 2.43-2.65 (m, 3H), 1.89 (m, 1H); ESI MS m/z for C₁₀H₁₁N₅ calculated: 201.10. found: 202.05 (M+H)⁺.

### Example 11

### (±)-9-(2,2-dimethyl-tetrahydro-3aH-cyclopenta[d][1,3]dioxol-4-yl)-9H-purin-6-amine

To a solution of 9-(cyclopent-2-enyl)-9H-purin-6-amine (36 mg, 0.18 mmol) in acetone-water (2 mL-1 mL) was added N-methylmorpholine-N-oxide NMO (42 mg, 0.36 mmol) followed by OsO₄ solution in water (0.1 mL, 0.008 mmol). The mixture was stirred at room temperature for 18 h. The reaction was quenched with 20% Na₂S₂O₅ solution (1 mL). The solvent was removed under reduced pressure. The residue was treated with MeOH and filtered with a pad of celite.

After filtration and concentration, the residue was dissolved in 8 mL acetone followed by 4 mL of 2,2-dimethoxypropane and catalytic amount of conc. H₂SO₄. The reaction was stirred at room temperature for 18 h. The reaction was quenched with TEA. The solvent was removed under reduced pressure. The crude products were purified by preparative TLC on silica gel (eluting with EtOAc/petroleum ether=90:10) to give 10 mg (0.036 mmol) of (3aS,4R,6aR)-isomer and 8 mg (0.029) of (3aR,4R,6aS)-isomer.

### (3aS,4R,6aR)-isomer

¹H NMR (CDCl₃, 400 MHz): δ 8.35 (s, 1H), 7.83 (s, 1H), 6.67 (brs, 2H), 4.96 (s, 2H), 4.86 (dd, J=7.6 Hz, 4.4 Hz, 1H), 2.53 (m, 1H), 2.13 (m, 3H), 1.53 (s, 3H), 1.34 (s, 3H); HR-MS (ES r) m/z for C₁₃H₁₈N₅O₂ calculated: 276.1455. found: 276.1444 (M+H)⁺.

### (3aR,4R,6aS)-isomer

¹H NMR (CDCl₃, 400 MHz): δ 8.31 (s, 1H), 8.25 (s, 1H), 4.81 (m, 2H), 4.69 (t, J=5.2 Hz, 1H), 2.39-2.28 (m, 1H), 2.17-2.07 (m, 2H), 1.53 (s, 3H), 1.29 (s, 3H); HR-MS (ESI⁺) m/z for C₁₃H₁₈N₅O₂ calculated: 276.1455. found: 276.1442 (M+H)⁺.

### Example 12

### (±)-(1R,2S,3R)-3-(6-Amino-9H-purin-9-yl)-1,2-cyclopentanediol hydrochloride

The same experimental procedure employed in the example 5. 9-((3aS,4R,6aR)-2,2-dimethyl-tetrahydro-3aH-cyclopenta[d][1,3]dioxol-4-yl)-9H-purin-6-amine gave 90% yield of the corresponding product. ¹H NMR (MeOD, 400 MHz): δ 8.425 (s, 1H), 8.36 (s, 1H), 4.51 (m, 1H), 4.18 (s, 1H), 2.50-2.40 (m, 1H), 2.35-2.26 (m, 1H), 2.18-2.09 (m, 1H), 1.88-1.80 (m, 1H); HR-MS (ESI⁺) m/z for C₁₀H₁₄N₅O₂ calculated: 236.1142. found: 236.1134 (M+H)⁺. for C₁₀H₁₃N₅NaO₂ calculated: 258.0962. found: 258.0955 (M+Na)⁺.

### Example 13

### (±)-(1R,2S,3S)-3-(6-Amino-9H-purin-9-yl)-1,2-cyclopentanediol hydrochloride

The same experimental procedure employed in the example 5. 9-((3aR,4R,6aS)-2,2-dimethyl-tetrahydro-3aH-cyclopenta[d][1,3]dioxol-4-yl)-9H-purin-6-amine gave 88% yield of the corresponding product. ¹H NMR (MeOD, 400 MHz): δ 8.57 (s, 1H), 8.39 (s, 1H), 5.13 (dd, J=14.8 Hz, 8.8 Hz, 1H), 4.26 (dd, J=9.5 Hz, 5.1 Hz, 1H), 4.17 (t, J=4.8 Hz, 1H), 2.35 (dd, J=16.4 Hz, 6.8 Hz, 2H), 2.04-1.93 (m, 2H); HR-MS (ESI⁺) m/z for C₁₀H₁₄N₅O₂ calculated: 236.1142. found: 236.1132 (M+H)⁺. for C₁₀H₁₃N₅NaO₂ calculated: 258.0962. found: 258.0949 (M+Na)⁺.

Biological studies imply that modifications of DZNep can lead to compounds which are almost equally effective as small molecule modulators of EZH2 complex and associated H3K27 trimethylation.

Therefore, the compounds based on the 3-Deazaneplanocin A (DZNep) core structure are valuable lead compounds for developing more potent anticancer compounds which target the Polycomb repressive complex 2 (PRC2) proteins. These compounds may be important as drugs on their own, or as an effective co-therapeutic as the lead compound, DZNep, has been shown to behave synergistically with histone deacetylase (HDAC) inhibitors to induce apoptosis of cancer cells through effective reversal of malignant chromatin modifications. This combination treatment has resulted in marked inhibition of Wnt/β-catenin signalling pathway in colon cancer cells suggesting that the combination of DZNep with HDAC inhibitors may provide an effective epigenetic treatment for human cancer.

### Structure of Compounds

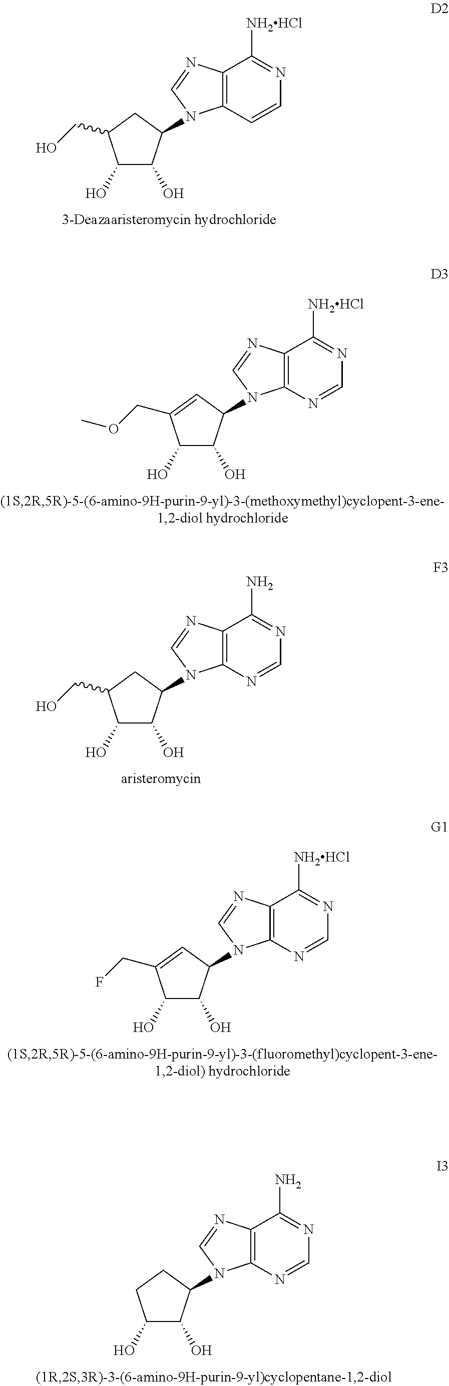

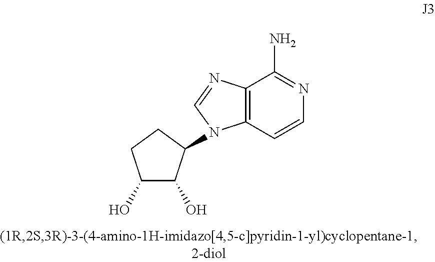

### Synthesis of Compounds

### Example 14

### Aristeromycin (F3)

The same experimental procedure used in the example 2 to obtain 4 mg (20% yield) of the title product (ratio of two diastereoisomers=2:1) from 20 mg (0.08 mmol) of neplanocin A. The title compound was purified by preparative LCMS. ESI MS m/z for C₁₁H₁₅N₅O₃ calculated: 265.12. found: 288.07 (M+Na)⁺.

### Example 15

### (1R,4R,5S)-9-N-[3-(fluoromethyl)-4,5-O,O-isopropylidene-2-cyclopenten-L-yl]-N⁶,N⁶-bis-(tert-butoxycarbonyl)adenine

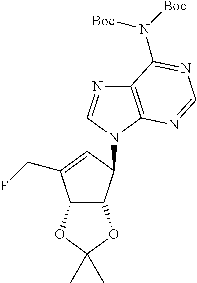

To a solution of (1R,4R,5S)-9-N-[3-(hydroxymethyl)-4,5-O,O-isopropylidene-2-cyclopenten-L-yl]-N⁶,N⁶-bis-(tert-butoxycarbonypadenine (20 mg, 0.02 mmol) in 1 mL of DCM was added slowly 8 μL of diethylaminosulphurtrifluoride (DAST) under the argon atmosphere at 0° C. The resultant mixture was stirred at room temperature for 3 h. Then the reaction was quenched with 1 mL of methanol. Evaporated the solvent and purified the product with flash chromatography. (eluent: 1% of methanol in DCM) Finally 14 mg (yield 70%) of white foam was obtain. ¹H NMR (CDCl₃, 400 MHz): δ 8.89 (s, 1H), 8.07 (s, 1H), 5.59 (s, 1H), 5.68 (s, 1H), 5.45 (d, J=5.6 Hz, 1H), 5.24 (s, 1H), 5.125 (s, 1H), 4.80 (d, J=5.2 Hz, 1H), 1.51 (s, 3H), 1.47 (s, 18H), 1.375 (s, 3H);

### Example 16

### (1S,2R,5R)-5-(6-amino-9H-purin-9-yl)-3-(fluoromethyl)cyclopent-3-ene-1,2-diol hydrochloride (G1)

The same experimental procedure employed in the example 5. tert-butyl-9-43aS,4R,6aR)-4,6a-dihydro-2,2-dimethyl-6-((trityloxy)methyl)-3aH-cyclopenta[d][1,3]dioxol-4-yl)-9H-purin-6-ylcarbamate gave 95% yield of the corresponding product. ¹H NMR (MeOD, 400 MHz): δ 8.38 (s, 1H), 8.36 (s, 1H), 6.04 (s, 1H), 5.62 (s, 1H), 5.20-5.07 (m, 2H), 4.68 (s, 1H), 4.43 (s, 1H); ESI MS m/z for C₁₁H₁₂FN₅O₂ calculated: 265.10. found: 266.06 (M+H)⁺.

### Example 17

### ((3aR,4R,6aR)-2,2-diethyl-6-methoxytetrahydrofuro[3,4-d][1,3]dioxol-4-yl)methanol

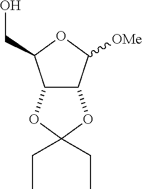

Concentrated hydrochloric acid (3.5 mL) was added to a suspension of D-ribose (35 g, 233 mmol) in 3-pentanone (140 mL) and methanol (140 mL) at room temperature. The mixture was refluxed for 6 h, cooled to room temperature, neutralized with saturated NaHCO₃ solution and partitioned between water (350 mL) and diethyl ether (100 mL). The separated aqueous phase was extracted with diethyl ether (2×100 mL) and ethyl acetate (3×100 mL). The combined organic phases were washed with water, brine prior, dried over MgSO₄. Removal of organic solvent under reduced pressure gave the title compound (37.9 g, 70%). ¹H NMR (CDCl₃, 400 MHz): δ 4.96 (s, 1H), 4.80 (d, 1H, J=6.0 Hz), 4.57 (d, 1H, J=6.0 Hz), 4.42 (dd, 1H, J=3.2, 2.8 Hz), 3.62 (m, 2H), 3.40 (s, 3H), 3.27 (dd, 1H, J=10.8, 2.8 Hz), 1.68 (q, 2H, J=7.6 Hz), 1.55 (q, 2H, J=7.6 Hz), 0.90 (t, 3H, J=7.6 Hz), 0.85 (t, 3H, J=7.6 Hz); HR-MS (ESI⁺) m/z for C₁₁H₂₀NaO₅ calculated 255.1208. found 255.1194 (M+Na)⁺.

### Example 18

### (3aS,4S,6aR)-2,2-diethyl-4-(iodomethyl)-6-methoxytetrahydrofuro[3,4-d][1,3]dioxole

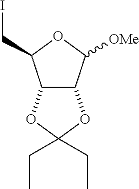

A solution of ((3aR,4R,6aR)-2,2-diethyl-6-methoxytetrahydrofuro[3,4-d][1,3]dioxol-4-yl)methanol (15.0 g, 64.6 mmol), imidazole (6.59 g, 96.9 mmol), and triphenylphosphine (20.3 g, 77.5 mmol) in toluene (250 mL) and acetonitrile (50 mL) was treated portionwise with iodine (19.7 g, 77.5 mmol), refluxed for 15 min, and cooled to room temperature. Additional iodine was introduced in approximately 100 mg portions until the reaction mixture remained dark-brown in color. After dilution with diethyl ether and repeated washing of the organic extracts with 10% sodium thiosulfate solution, water, and brine, the solution was dried with MgSO₄, filtered and concentrated under reduced pressure. The residue was filtered through a short pad of silica gel (elution with 95:5 heptane-ethyl acetate) to give the title compound (20.1 g, 91%) as a colorless oil. ¹H NMR (CDCl₃, 400 MHz): δ 5.06 (s, 1H), 4.76 (d, 1H, J=6.0 Hz), 4.63 (d, 1H, J=6.0 Hz), 4.46 (dd, 1H, J=10.0, 6.4 Hz), 3.38 (s, 3H), 3.28 (dd, 1H, J=10.0, 6.4 Hz), 3.17 (t, 1H, J=10.0 Hz), 1.70 (q, 2H, J=7.6 Hz), 1.58 (q, 2H, J=7.6 Hz), 0.91 (t, 3H, J=7.6 Hz), 0.88 (t, 3H, J=7.6 Hz); HR-MS (ESI⁺) m/z for C₁₁H₁₉INaO₄ calculated 365.0226. found 365.0213 (M+Na)⁺.

### Example 19

### (4R,5R)-2,2-diethyl-5-vinyl-1,3-dioxolane-4-carbaldehyde

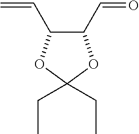

Powdered zinc metal (16 g, 245.5 mmol) was added to a solution of (3aS,4S,6aR)-2,2-diethyl-4-(iodomethyl)-6-methoxytetrahydrofuro [3,4-d][1,3]dioxole (16.8 g, 49.1 mmol) in methanol (100 mL), and the mixture was refluxed for 1 h, cooled, and filtered. The filtrate was concentrated under reduced pressure at 30° C., and the residue was quickly purified on silica gel column (elution with 4:1 heptane-ethyl acetate) to afford the title compound (7.24 g, 80%) as a homogeneous colorless oil. ¹H NMR (CDCl₃, 400 MHz): δ 9.55 (d, 1H, J=3.2 Hz), 5.74 (ddd, 1H, J=17.2, 10.4, 6.8 Hz), 5.45 (dt, 1H, J=17.2, 1.2 Hz), 5.30 (dt, 1H, J=10.4, 1.2 Hz), 4.87 (dd, 1H, J=8.0, 6.8 Hz), 4.41 (dd, 1H, J=8.0, 3.2 Hz), 1.84 (q, 2H, J=7.6 Hz), 1.69 (q, 211, J=7.6 Hz), 1.02 (t, 3H, J=7.6 Hz), 0.94 (t, 3H, J=7.6 Hz); HR-MS (ESI⁺) m/z for C₁₀H₁₆NaO₃ calculated 207.0997. found 207.0985 (M+Na)⁺.

### Example 20

### (3aR,6aR)-2,2-diethyl-3aH-cyclopenta[d][1,3]dioxol-4(6aH)-one

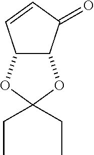

To a solution of (4R,5R)-2,2-diethyl-5-vinyl-1,3-dioxolane-4-carbaldehyde (4.0 g, 21.71 mmol) in anhydrous DCM (75 mL) was added dropwise a solution of vinylmagnesium bromide (0.7 M in THF, 37.2 mL) at 0° C. The reaction was allowed to warm to room temperature and stirred for 18 h. Saturated NH₄Cl (20 mL) was added to quench the reaction. The organic layer was separated, washed with brine, dried over MgSO₄ and filtered. The solvent was removed under reduced pressure to give the crude residue which was redissolved in anhydrous DCM (100 mL). To this solution was added Hoveyda-Grubbs 2^{nd} generation catalyst, (150 mg, 0.24 mmol) and the reaction mixture was allowed to stir under argon atmosphere for 3 h. Pyridinium chlorochromate (PCC) (9.36 g, 43.42 mmol) was then added. The reaction mixture was stirred for another 3 h, and then filtered over a short pad of celite/florisil (elution with ethyl acetate). The combined organic layers were evaporated to dryness, then purified by a flash chromatography on silica gel (elution with 9:1 heptane-ethyl acetate) to give the title compound (2.15 g, 54% over three-step sequence) as a white amorphous solid. ¹H NMR (CDCl₃, 400 MHz): δ 7.58 (dd, 1H, J=6.0, 1.6 Hz), 6.19 (d, 1H, J=6.0 Hz), 5.27 (dd, 1H, J=4.8, 1.6 Hz), 1.66 (q, 2H, J=7.6 Hz), 1.61 (q, 2H, J=7.6 Hz), 0.91 (t, 3H, J=7.6 Hz), 0.80 (t, 3H, J=7.6 Hz); HR-MS (ESI⁺) m/z for C₁₀H₁₄NaO₃ calculated 205.0841. found 205.0832 (M+Na)⁺.

### Example 21

### (3aS,4S,6aR)-2,2-diethyl-4,6a-dihydro-3aH-cyclopenta[d][1,3]dioxol-4-ol

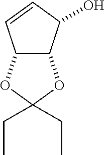

The 3aR,6aR)-2,2-diethyl-3aH-cyclopenta[d][1,3]dioxol-4(6aH)-one (2.0 g, 10.98 mmol) and CeCl₃.7H₂O (4.1 g, 10.98 mmol) were added to MeOH (100 mL) cooled to 0° C., then NaBH₄ (0.42 g, 12.6 mmol) was added portionwise. The mixture was stirred at this temperature for 20 min then quenched with water (100 mL). The mixture was extracted with DCM (3*100 mL), and then the combined organic layers were washed with brine. After drying over MgSO₄ and filtration, the solvent was removed under reduced pressure. Flash chromatography on silica gel (elution with 9:1 heptane-ethyl acetate) gave the title compound (1.92 g, 95%) as a colorless liquid. ¹H NMR (CDCl₃, 400 MHz): δ 5.87 (s, 2H), 5.03 (d, 1H, J=5.6 Hz), 4.74 (t, 1H, J=5.6 Hz), 4.55 (dd, 1H, J=10.0, 5.6 Hz), 2.78 (d, 1H, J=10 Hz), 1.67 (m, 4H), 0.92 (t, 3H, J=7.6 Hz), 0.87 (t, 3H, J=7.6 Hz); HR-MS (ESI⁺) m/z for C₁₀H₁₆NaO₃ calculated 207.0997. found 207.0982 (M+Na)⁺.

### Example 22

### (3aR,4S,6aR)-2,2-diethyl-4,6a-dihydro-3aH-cyclopenta[d][1,3]dioxol-4-yl 4-methylbenzenesulfonate

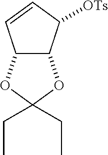

To a solution of (3aS,4S,6aR)-2,2-diethyl-4,6a-dihydro-3aH-cyclopenta[d][1,3]dioxol-4-ol (1.50 g, 8.14 mmol) and p-toluenesulfonyl chloride (3.1 g, 16.28 mmol) in DCM (30 mL) was added Et₃N (0.46 g, 4.5 mL, 32.56 mmol). The mixture was stirred for 24 h at room temperature under argon atmosphere. The mixture was extracted with H₂O (10 mL) and brine (10 mL). The organic layer was dried over MgSO₄, filtered, and concentrated to dryness. Flash chromatography purification on silica gel (elution with 4:1 heptane-ethyl acetate) furnished the title compound (2.26 g, 82%) as a white amorphous solid. ¹H NMR (CDCl₃, 400 MHz): δ 7.87 (d, 21-1, J=8.4 Hz), 7.33 (d, 211, J=8.4 Hz), 6.01 (m, 1H), 5.77 (m, 1H), 5.22 (m, 1H), 4.95 (d, 1H, J=5.6 Hz), 4.83 (t, 1H, J=5.6 Hz), 2.45 (s, 3H), 1.55 (m, 4H), 0.78 (m, 6H); HR-MS (ESI⁺) m/z for C₁₇H₂₂NaO₅S calculated 361.1086. found 361.1078 (M+Na)⁺.

### Example 23

### (1S,2R,5R)-5-(6-amino-9H-purin-9-yl)cyclopent-3-ene-1,2-diol

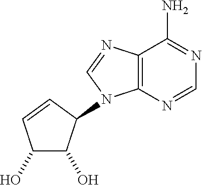

To a solution of (3aS,4S,6aR)-2,2-diethyl-4,6a-dihydro-3aH-cyclopenta[d][1,3]dioxol-4-ol (110 mg, 0.597 mmol), N⁶,N⁶-Bis-(tert-butoxycarbonypadenine (239 mg, 0.716 mmol, prepared according to the Tetrahedron 2007, 63, 9836-9841) and triphenylphosphine (266 mg, 1.015 mmol) in anhydrous THF (3 mL) was added dropwise the diisopropyl azodicarboxylate (DIAD, 181 mg, 0.896 mmol) at 0° C. The reaction was allowed to stir for 18 h at room temperature. The solvent was removed under reduced pressure. The partial purification of the crude by using flash chromatography on silica gel (elution 2% methanol in DCM) gave the expected Mitsunobu adduct together with hydrazine (derivated from DIAD), which was used in the next step of synthesis. To this mixture was added in MeOH (1 mL) and 10% hydrochloric acid solution (1 mL). The resultant mixture was stirred at room temperature for 2 h. The reaction was then diluted with water (5 mL). The aqueous layer was washed with DCM (3*2 mL) and evaporated to dryness under reduced pressure. The residue was redissolved in MeOH (3 mL). The solid NaHCO₃ (100 mg) was added and stirred at room temperature for 5 min. After filtration, the filtrate was added silica gel (300 mg) and evaporated under reduced pressure. The crude was purified by flash chromatography on silica gel (elution with 10% of methanol in DCM) to give the title compound (84 mg, 60% over 2 steps) as a white amorphous solid. ¹H NMR (CD₃OD, 400 MHz): δ 8.21 (s, 1H), 8.14 (s, 1H), 6.28 (m, 1H), 6.14 (dd, 1H, J=6.4, 1.5 Hz), 5.56 (m, 1H), 4.72 (m, 1H), 4.41 (t, 1H, J=5.6 Hz) ppm; HR-MS (ESI⁺) m/z for C₁₀H₁₂N₅O₂ calculated 234.0991. found 234.0980 (M+H)⁺. for C₁₀H₁₁N₅NaO₂ calculated 256.0810. found 256.0780 (M+Na)⁺.

### Example 24

### (1R,2S,3R)-3-(6-amino-9H-purin-9-yl)cyclopentane-1,2-diol (I3)

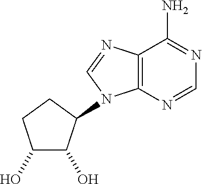

A solution of (1S,2R,5R)-5-(6-amino-9H-purin-9-yl)cyclopent-3-ene-1,2-diol (30 mg, 0.129 mmol) in MeOH (1 mL) was added into a round bottom flask containing palladium on charcoal (10%, 5 mg). The suspension was allowed to stir under hydrogen atmosphere for 18 h, and then filtered. To the obtained solution was added silica gel (40 mg) and the solvent was removed under reduced pressure. The residue was purified by flash chromatography on silica gel (elution 10% of MeOH in DCM) to give the title compound (20 mg, 75%) as a white amorphous solid. ¹H NMR (CD₃OD, 400 MHz): δ 8.23 (s, 1H), 8.20 (s, 1H), 4.85 (m, 1H), 4.55 (dd, 1H, J=9.2, 4.4 Hz), 4.21 (td, 1H, J=4.8, 2.0 Hz), 2.42 (m, 1H), 2.32 (m, 1H), 2.16 (m, 1H), 1.84 (m, 1H) ppm; HR-MS (ESI⁺) m/z for C₁₀H₁₄N₅O₂ calculated 236.1147. found 236.1135 (M+H)⁺. for C₁₀H₁₃N₅NaO₂ calculated 258.0967. found 258.0951 (M+Na)⁺.

### Example 25

### 1-((3aS,4R,6aR)-2,2-diethyl-4,6a-dihydro-3aH-cyclopenta[d][1,3]dioxol-4-yl)-1H-imidazo[4,5-c]pyridin-4-amine

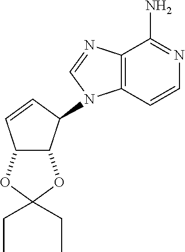

To a solution of 3-deaazaadenine (59.5 mg, 0.443 mmol, prepared according to the literature procedure, Bioorg. Med. Chem. 2006, 14, 1935-1941) in anhydrous DMF (3 mL) at 0° C. was added sodium hydride (60%, 17.8 mg, 0.443 mmol). The mixture was stirred for 5 min at room temperature, then a solution of (3aR,4S,6aR)-2,2-diethyl-4,6a-dihydro-3aH-cyclopenta[d][1,3]dioxol-4-yl 4-methylbenzenesulfonate (100 mg, 0.295 mmol) in anhydrous DMF (1 mL) was added. The reaction mixture was allowed to stir at 55° C. for 36 h, and then the solvent was removed under reduced pressure. DCM (15 mL) was added and the mixture was sonicated for 5 min, filtered and evaporated under reduced pressure. Flash chromatography on silica gel (elution with 5% MeOH in DCM) gave the title compound (49.7 mg, 56%) as a white amorphous solid. ¹H NMR (CDCl₃, 400 MHz): δ 7.88 (d, 1H, J=5.6 Hz), 7.66 (s, 1H), 6.81 (d, 1H, J=5.6 Hz), 6.37 (d, 1H, J=5 Hz), 6.07 (d, 1H, J=5 Hz), 5.43 (m, 1H), 5.37 (s, 1H), 5.30 (bs, 2H), 4.58 (d, 1H, J=5.2 Hz), 1.71 (q, 2H, J=7.6 Hz), 1.61 (q, 2H, J=7.6 Hz), 0.92 (t, 3H, J=7.6 Hz), 0.88 (t, 3H, J=7.6 Hz); HR-MS (ESI⁺) m/z for C₁₆H₂₁N₄O₂ calculated 301.1665. found 301.1657 (M+H)⁺. for C₁₆H₂₀N₄NaO₂ calculated 323.1484. found 323.1496 (M+Na)⁺.

### Example 26

### (1S,2R,5R)-5-(4-amino-1H-imidazo[4,5-c]pyridin-1-yl)cyclopent-3-ene-1,2-diol

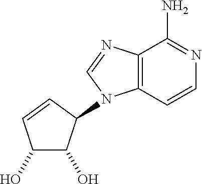

To a solution of 1-((3aS,4R,6aR)-2,2-diethyl-4,6a-dihydro-3aH-cyclopenta[d][1,3]dioxol-4-yl)-1H-imidazo[4,5-c]pyridin-4-amine (45 mg, 0.150 mmol) in MeOH (1 mL) was added hydrochloric acid aqueous solution (10%, 1 mL). The mixture was stirred at room temperature for 2 h, and then water (5 mL) was added. This aqueous phase was washed 3 times with CH₂Cl₂ then evaporated to dryness under reduced pressure. The obtained residue was dissolved in MeOH (2 mL) and solid NaHCO₃ (50 mg) was added. The mixture was stirred at room temperature for 5 min, and filtered. To this solution was added silica gel (60 mg) and the solvent was removed under reduced pressure. The residue was purified by flash chromatography on silica gel (elution with 10% MeOH in DCM) to give the title compound (33 mg, 95%) as a white amorphous solid. ¹H NMR (CD₃OD, 400 MHz): δ 8.13 (s, 1H), 7.66 (d, 1H, J=6.4 Hz), 6.99 (d, 1H, J=6.4 Hz), 6.34 (m, 1H), 6.20 (dd, 1H, J=6.4, 1.6 Hz), 5.44 (dd, 1H, J=3.6, 1.5 Hz), 4.67 (m, 1H), 4.22 (t, 1H, J=5.6 Hz); HR-MS (ESI⁺) m/z for C₁₁H₁₃N₄O₂ calculated 233.1039. found 233.1033 (M+H)⁺.

### Example 27

### (1R,2S,3R)-3-(4-amino-1H-imidazo[4,5-c]pyridin-1-yl)cyclopentane-1,2-diol (J3)

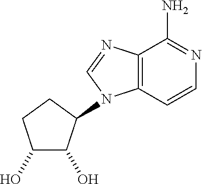

A solution of (1S,2R,5R)-5-(4-amino-1H-imidazo[4,5-c]pyridin-1-yl)cyclopent-3-ene-1,2-diol (30 mg, 0.129 mmol) in MeOH (1 mL) was added into a round bottom flask containing palladium on charcoal (10%, 5 mg). The suspension was allowed to stir under hydrogen atmosphere for 18 h, and then filtered. To the obtained solution was added silica gel (40 mg) and the solvent was removed under reduced pressure. The residue was purified by flash chromatography on silica gel (elution with 10% of MeOH in DCM) to give the title compound (20 mg, 66%) as a white amorphous solid. ¹H NMR (CD₃OD, 400 MHz): δ 8.35 (s, 1H), 7.67 (d, 1H, J=6.8 Hz), 7.08 (d, 1H, J=6.8 Hz), 4.80 (q, 1H, J=9.2 Hz), 4.35 (dd, 1H, J=9.2, 4.4 Hz), 4.19 (td, 1H, J=4.8, 2.0 Hz), 2.48 (m, 1H), 2.31 (m, 1H), 2.04 (m, 1H), 1.86 (m, 1H); HR-MS (ESI⁺) m/z for C₁₁H₁₅N₄O₂ calculated 235.1195. found 235.1190 (M+H)⁺. for C₁₁H₁₄N₄NaO₂ calculated 257.1014. found 257.1006 (M+Na)⁺.

### In Vitro and In Vivo Investigations of DZNep-Like Compounds as Novel Anti-Tumor Drugs.

Fluorescence activated cell sorting (FACS) also known as flow cytometry is the technique used to determine apoptosis in this set of experiments. Apoptosis is indicated by cell populations with DNA content in the subG1 range. To determine the activity of these compounds, the inventors used HCT115 ER-E2F1 cells to determine the activity of the compound in inducing E2F1-dependent apoptosis. In this assay, addition of 4-OHT ligand will activate E2F1. DZNep has been show previously to activate OHT-induced apoptosis in this system.

### Results

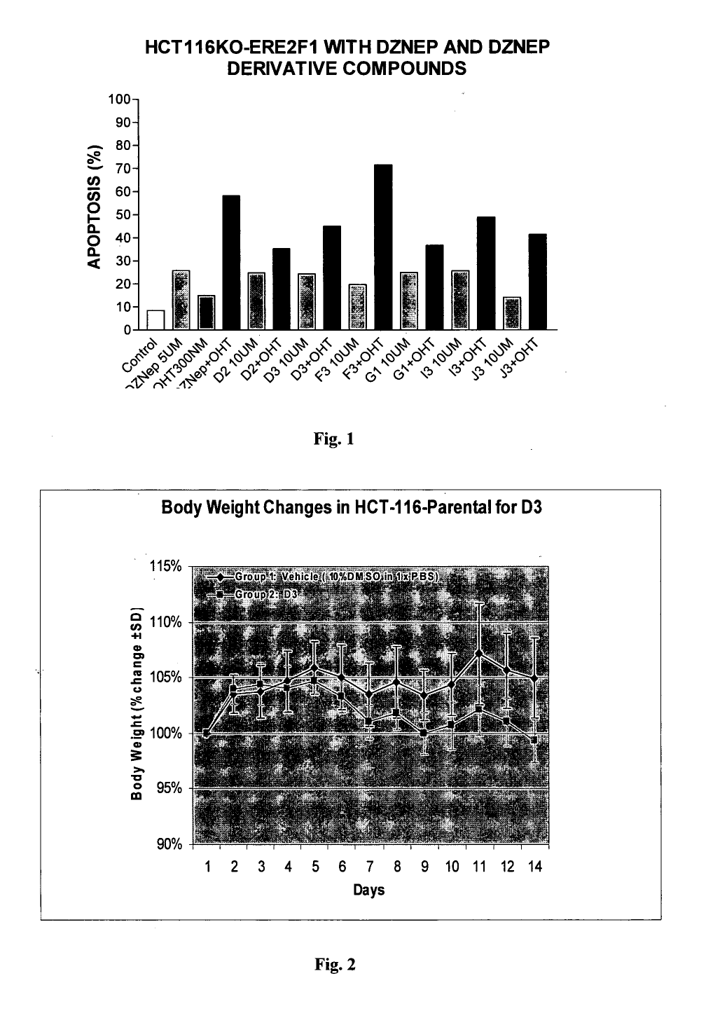

From preliminary results obtained, 6 candidate compounds; D2, D3, F3, G1, I3 and J3 have been identified to show similar activities with DZNep, namely they can induce E2F1-dependent apoptosis upon OHT treatment. D3 shows an ability to cause apoptosis as effectively as DZNep in the induction of E2F1-dependent apoptosis. In vivo experiments show that D3 has a higher maximum tolerated dose (MTD) as compared to DZNep. Results are shown graphically in FIG. 1.

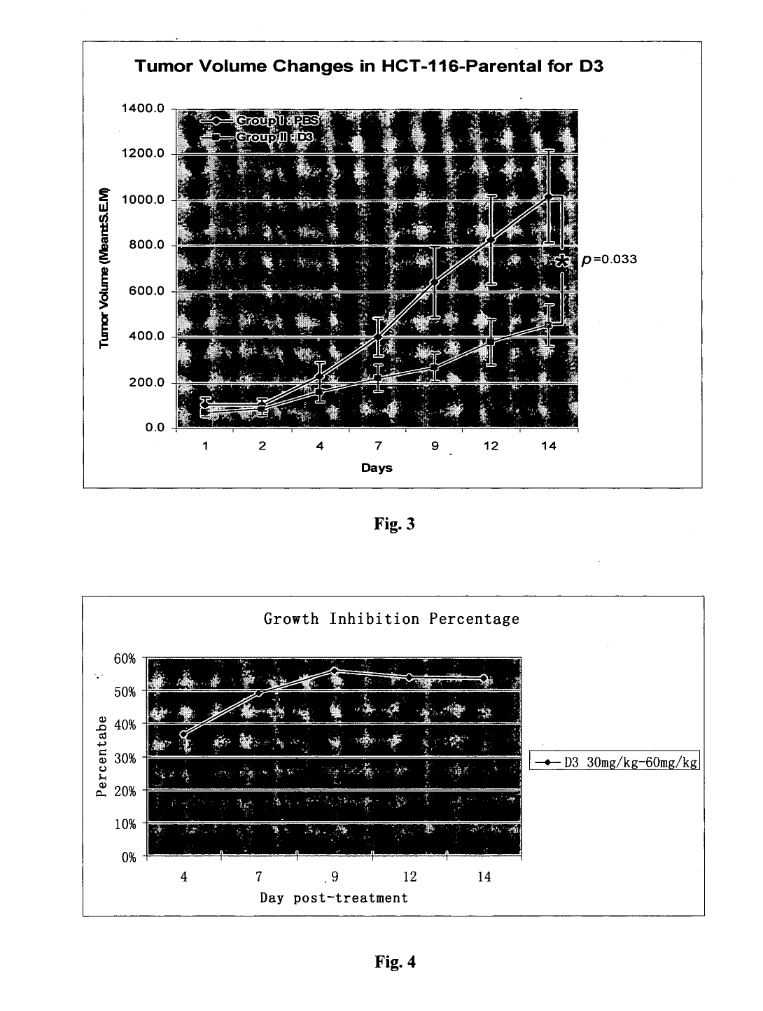

In vivo tumor xenograft work was conducted to determine the anti-tumor activity of one of the compounds, D3. Results are shown in FIGS. 2 to 4, showing, respectively, body weight changes, tumour volume changes and growth inhibition.

### In Vivo Efficacy of D3 in a Mouse HCT-116 Colorectal Carcinoma Xenograft Model

The in vivo efficacy and toxicity of D3 was evaluated in a mouse HCT-116 colorectal carcinoma xenograft model.

Female athymic BALB/c nude mice (ARC, West Australia), 18-20 weeks of age, are fed with sterilized tap water (ad libitum water) and irradiated standard rodent diet consisting of: 19% protein, 5% fat, and 5% fiber. Mice are housed in individual ventilated cages on 12-hour light cycle at 21-22° C. and 40-60% humidity. Biological Resource Centre, Biopolis (BRC) complies with the recommendations of the guide for care and use of Laboratory Animals with respect to restraint, husbandry, surgical procedures, feed and fluid regulation, and veterinary care. The animal cares and use program (#070276) at BRC was Institutional Animal Care and Use Committee (IACUC) accredited.

D3 was provided in powder form, dissolved in 10% DMSO in sterile 1×PBS and stored in −20° C. Every mouse will receive a dose of 30-60 mg per kilogram body weight by intraperitoneal injection (ip).

### Tumor Implantation

Mice were implanted subcutaneously in the left flank with 5×10⁶ cells of HCT-116 parental human colon carcinoma. The tumors were allowed to grow for 8 days and thereafter monitored every 2-3 days by caliper.

### Treatment Plan

On the day 1, nude mice were divided into 2 groups according to tumor volume to ensure tumor volume evenly distributed into each group. Each group comprised 7 animals. Drug treatment was initiated on day 1. D3 is administered ip at doses of 30 mg/kg for 7 days followed by 60 mg/kg for another 5 days on once-a-day basis. Another group of mice received receive only vehicle by ip route. 13 was given 30 mg/kg and 60 mg/kg, respectively. The study was terminated on Day 14.

Estimated tumor volume was calculated using the formula:

Tumor volume (mm³)=(w²×l)/2

where w=width and l=length in mm of an HCT-116 carcinoma.

### Efficacy Evaluation

Compound efficacy was assessed by the tumor growth inhibition (TGI) method in which tumor volume of treatment groups are compared to the vehicle controls. The percent tumor growth inhibition (% TGI) is calculated as follows:

% TGI=(C_{day a}−T_{day a})(C_{day a}−C_{day 1})×100

where:
C_{day 1}=median tumor volume for the control group (vehicle) on day 1
C_{day a}=median tumor volume for the control group (vehicle) on day a
T_{day a}=median tumor volume for a treatment group on day a
   Animals classified as NTRD (non-treatment-related deaths) were excluded from TGI calculations.

### Toxicity and Endpoints

Animals were weighed daily from day 1. Mice were examined frequently for clinical signs of any adverse, drug-related side effects, including activity (inactivity/hyperactivity), skin hydration/dehydration, posture (for example hunched), gait, seizure when put on weighing scale, body temperature (for example, cool to touch) and vocalization.

### Statistical Analysis

The two sample t-test was used to determine the statistical significance of body weight changes and tumor volumes between groups. Statistical analyses were conducted at a p level of 0.05. SPSS was used for all statistical analyses and graphic presentations.

### Result and Discussion

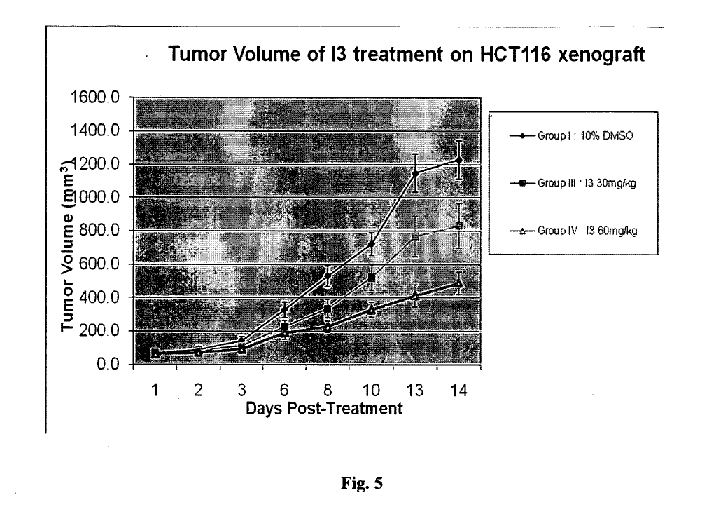

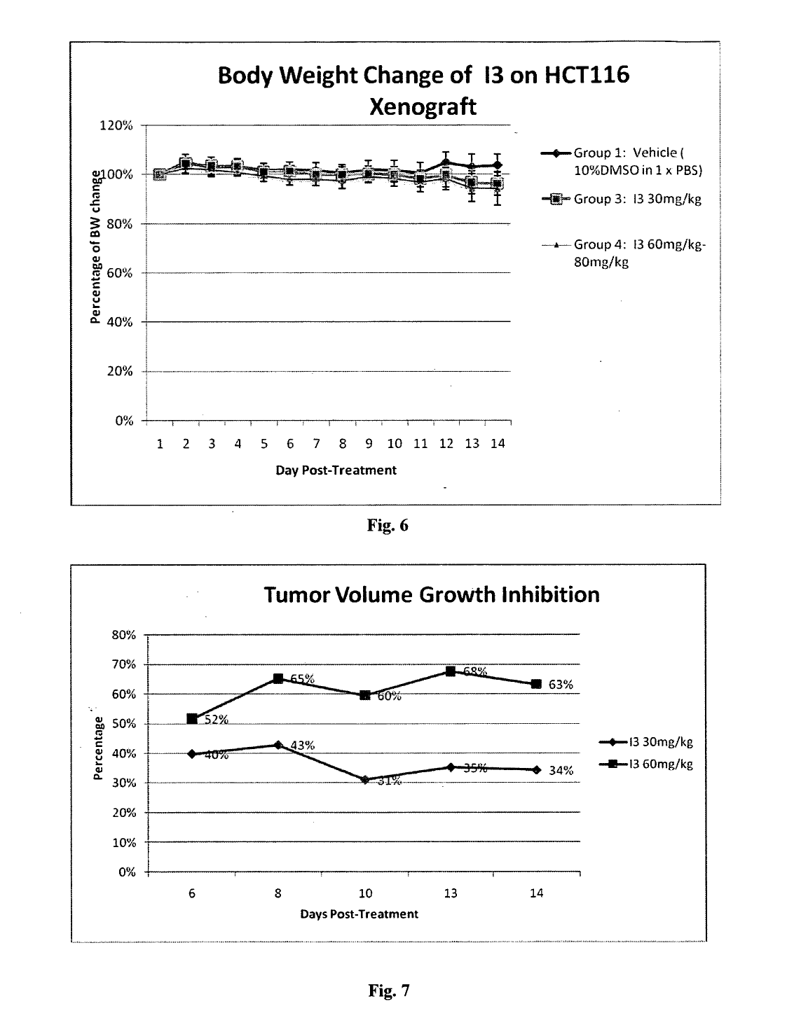

D3: No obvious body weight loss was observed during the whole procedure period (>95% of original body weight) and tumor volume of D3 group was statistically significantly smaller than that of vehicle group (p=0.033). As to the tumor growth inhibition, at the time point of day 7, 9, 12 and end point of the day 14, tumor growth inhibition is about 49%, 56%, 54% and 54% respectively. See FIGS. 2-4.
I3: No obvious body weight loss was observed during the whole procedure period (>95% of original body weight) and Tumor volume of I3 at 30 mg/kg group and 60 mg/kg was statistically significantly smaller than that of vehicle group (p=0.037 and p=0.000 respectively). As to the tumor growth inhibition for I3 at doses of 30 mg/kg, at the time point of day 6, 8, 10, 13 and end point of the day 14, tumor growth inhibition is about 40%, 43%, 31%, 35% and 34% respectively. As for I3 at doses of 60-80 mg/kg, at the time point of day 6, 8, 10, 13 and end point of the day 14, tumor growth inhibition is about 50%, 65%, 60%, 68% and 63% respectively. (see FIGS. 5-7)

Thus the invention relates to an anti-cancer compound for inhibiting the function of Polycomb repressive complex 2 (PRC2) proteins having the structure:

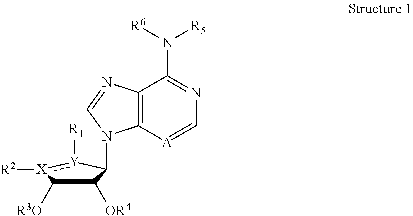

In particular embodiments of the compound A is independently carbon or nitrogen; X and Y are independently carbon and the bond between X and Y may be saturated or unsaturated; R¹ and R² are independently hydrogen or halogen or carbon or aliphatic, arylaliphatic, hydrocarbyl group comprising 1-8 main chain carbon atoms and 0-3 heteroatoms being N, O, S, Si, or a halogen such as Cl or F; R³, R⁴, R⁵ and R⁶ are independently hydrogen or aliphatic, cycloaliphatic, aromatic, arylaliphatic or arylcycloaliphatic hydrocarbyl groups, that comprise 0-3 heteroatoms being N, O, S, or Si; R³ and R⁴ may optionally be linked so as to define an aliphatic hydrocarbyl bridge. The invention also relates to an anti-cancer compound for inhibiting the function of Polycomb repressive complex 2 (PRC2) proteins having the structure:

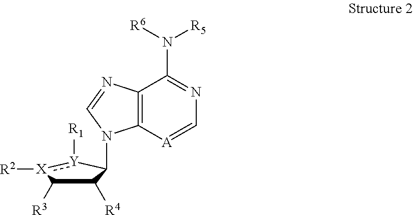

In particular embodiments of this compound A is independently carbon or nitrogen; X and Y are independently carbon and the bond between X and Y may be saturated or unsaturated; R¹ and R² are independently hydrogen or halogen or carbon or aliphatic, arylaliphatic, hydrocarbyl group comprising 1-8 main chain carbon atoms and 0-3 heteroatoms being N, O, S, Si, or a halogen such as Cl or F; R³ and R⁴ are independently hydrogen or halogen or carbon or aliphatic, cylcoaliphatic, aromatic, arylcycloaliphatic or arylaliphatic hydrocarbyl group; R⁵ and R⁶ are, independently hydrogen or aliphatic, cycloaliphatic, aromatic, arylaliphatic, or arylcycloaliphatic hydrocarbyl groups, that comprise 0-3 heteroatoms being N, O, S, or Si.

1. A compound of structure I: 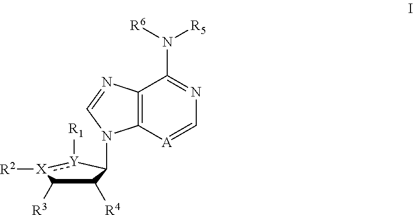
wherein:
X and Y are independently C or O,
A is C or N; [symbol]
is a single bond or a double bond;
R1 and R2 are either absent or independently selected from the group consisting of hydrogen, halogen, optionally substituted alkyl, optionally substituted aryl, optionally substituted alkyl-Z— and optionally substituted aryl-Z—, where Z is N, O, S or Si, or R1 and R2 together form an optionally substituted hydrocarbon bridge or an optionally substituted α,ω-dioxahydrocarbon bridge between X and Y;
R3 and R4 are independently selected from the group consisting of hydrogen, halogen, optionally substituted alkyl, optionally substituted aryl, optionally substituted alkyl-Z′— and optionally substituted aryl-Z′—, where Z′ is N, O, S or Si, or R3 and R4 together form an optionally substituted hydrocarbon bridge or an optionally substituted α,ω-dioxahydrocarbon bridge between the two carbon atoms to which they are attached;
R5 and R6 are independently selected from the group consisting of hydrogen, optionally substituted alkyl and optionally substituted aryl, or R5 and R6 together with the nitrogen atom to which they are attached form an optionally substituted azacycloalkyl group; or an enantiomer or diastereomer thereof or a salt of any of these,
wherein if either X or Y or both is O, [symbol] is a single bond and if X═O, R2 is absent and if Y═O, R1 is absent, and
wherein said compound is not 3-deazaneplanocin A or aristeromycin or 3-deazaaristeromycin hydrochloride or (1S,2R,5R)-5-(6-amino-9H-purin-9-yl)-3-(methoxymethyl)cyclopent-3-ene-1,2-diol hydrochloride or (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol or (1R,2S,3R)-3-(4-amino-1H-imidazo[4,5-c]pyridin-1-yl)cyclopentane-1,2-diol or (±)-(1R,2S,3R)-3-(6-amino-9H-purin-9-yl)-1,2-cyclopentanediol hydrochloride or 2′,3′-O-isopropylidene-3-deazaneplanocin A or (1S,2R,5R)-5-(6-amino-9H-purin-9-yl)-3-methylcyclopent-3-ene-1,2-diol hydrochloride.

2. The compound of claim 1 wherein:
X and Y are both C;
R¹ and R² are independently hydrogen, halogen, an aliphatic, arylaliphatic or hydrocarbyl group having between 1 and 8 main chain carbon atoms and between 0 and 3 heteroatoms, each heteroatom, independently, being N, O, S, Si (wherein if the heteroatom is N or Si, the other group(s) attached thereto are, independently, hydrogen, aryl or aliphatic);
R³ and R⁴ are independently hydroxy, alkoxy, cycloalkyloxy, aryloxy, arylalkoxy or arylcycloaalkyloxy groups comprising between 0 and 3 heteroatoms, each heteroatom, independently, being N, O, S, or Si (wherein if the heteroatom is N or Si, the other group(s) attached thereto are, independently, hydrogen, aryl or aliphatic) or R³ and R⁴ are linked so as to define an α,ω-dioxahydrocarbon bridge between the two carbon atoms to which they are attached; and
R⁵ and R⁶ are independently hydrogen, aliphatic, cycloaliphatic, aromatic, arylaliphatic or arylcycloaliphatic hydrocarbyl groups comprising between 0 and 3 heteroatoms, each heteroatom, independently, being N, O, S, or Si (wherein if the heteroatom is N or Si, the other group(s) attached thereto are, independently, hydrogen, aryl or aliphatic).

3. The compound of claim 1 wherein:
X and Y are both C;
R¹ and R² are independently hydrogen or halogen or carbon or aliphatic, arylaliphatic, hydrocarbyl group comprising 1-8 main chain carbon atoms and 0-3 heteroatoms being N, O, S, Si (wherein if the heteroatom is N or Si, the other group(s) attached thereto are, independently, hydrogen; aryl or aliphatic), or a halogen such as Cl or F;
R³ and R⁴ are independently hydrogen or halogen or carbon or aliphatic, cycloaliphatic, aromatic, arylcycloaliphatic or arylaliphatic hydrocarbyl group or are linked so as to define an aliphatic hydrocarbyl bridge; and
R⁵ and R⁶ are, independently hydrogen or aliphatic, cycloaliphatic, aromatic, arylaliphatic, or arylcycloaliphatic hydrocarbyl groups, that comprise 0-3 heteroatoms being N, O, S, or Si (wherein if the heteroatom is N or Si, the other group(s) attached thereto are, independently, hydrogen, aryl or aliphatic).

4. The compound of claim 1 wherein X and Y are both C.

5. The compound of claim 1 wherein R¹ is H.

6. The compound of claim 1 wherein R³ and R⁴ are both OH or together form a protected vicinal diol.

7. The compound of claim 1 wherein R³ and R⁴ together form an —OC(Me₂)O— group.

8. The compound of claim 1 which is ((3R,4S,5R)-3-(6-amino-9H-purin-9-yl)-4,5-dihydroxycyclopent-1-enyl)methyl benzoate hydrochloride.

9. The compound of claim 1, or an enantiomer, diastereomer or pharmaceutically acceptable salt thereof, showing activity to activate E2F1-induced apoptosis of at least about 15%.

10. The compound of claim 1, or an enantiomer, diastereomer or pharmaceutically acceptable salt thereof, showing activity to activate E2F1-induced apoptosis in the presence of 4-OHT of at least about 25%.

11. The compound of claim 1, or an enantiomer, diastereomer or pharmaceutically acceptable salt thereof, showing apoptosis induction in colon cancer cells with histone deacetylase inhibitor TSA of at least about 40%.

12. The compound of claim 1, or an enantiomer, diastereomer or pharmaceutically acceptable salt thereof, which is capable of inhibiting the function of Polycomb repressive complex 2 (PRC2) proteins.

13-17. (canceled)

18. A pharmaceutical composition comprising a compound according to claim 1, or an enantiomer, diastereomer or pharmaceutically acceptable salt thereof, together with one or more pharmaceutically acceptable carriers, diluents, excipients or adjuvants.

19. A method of treating cancer comprising administering to a patient in need thereof a clinically effective amount of a compound of structure I as defined in claim 1, or an enantiomer, diastereomer or pharmaceutically acceptable salt thereof, or of a pharmaceutical composition comprising a compound of structure I, or an enantiomer, diastereomer or pharmaceutically acceptable salt thereof, together with one or more pharmaceutically acceptable carriers, diluents, excipients or adjuvants.

20. The method of claim 19 wherein the compound is aristeromycin, 3-deazaaristeromycin hydrochloride, (1S,2R,5R)-5-(6-amino-9H-purin-9-yl)-3-(methoxymethyl)cyclopent-3-ene-1,2-diol hydrochloride, (1S,2R,5R)-5-(6-amino-9H-purin-9-yl)-3-(fluoromethyl)cyclopent-3-ene-1,2-diol) hydrochloride or (1R,2S,3R)-3-(6-amino-9H-purin-9-yl)cyclopentane-1,2-diol, (1R,2S,3R)-3-(4-amino-1H-imidazo[4,5-c]pyridin-1-yl)cyclopentane-1,2-diol, (1R,2S,3R)-3-(6-amino-9H-purin-9-yl)-1,2-cyclopentanediol hydrochloride, 2′,3′-O-isopropylidene-3-deazaneplanocin A or (1S,2R,5R)-5-(6-amino-9H-purin-9-yl)-3-methylcyclopent-3-ene-1,2-diol hydrochloride or an enantiomer or diastereomer thereof or a salt (e.g. a pharmaceutically acceptable salt) of any of these.
